(19) United States
(12) Patent Application Publication
(10) Pub. No.: US 2018/0354163 A1
(43) Pub. Date: Dec. 13, 2018

(54) METHOD OF MANUFACTURING KINESIO BRACE AND KINESIO BRACE

(71) Applicant: YU-CHIEN WANG, Taichung City (TW)

(72) Inventor: YU-CHIEN WANG, Taichung City (TW)

(21) Appl. No.: 16/100,588

(22) Filed: Aug. 10, 2018

Related U.S. Application Data
(63) Continuation-in-part of application No. 15/620,315, filed on Jun. 12, 2017.

(51) Int. Cl. B29C 37/00; B29C 33/40; A61F 5/01

(52) U.S. Cl. CPC B29C 37/0028; B29C 37/0053; A61F 5/0109; B29C 33/40

(57) ABSTRACT

The method of manufacturing kinesio brace includes steps of: preparing an elastic body which is tube-shaped to have a first face inside and a second face outside, the elastic body defining an axial direction; inserting an inner mold into the elastic body so that the first face faces to the inner mold; placing the inner mold with the elastic body into an outer mold and closing the outer mold, the outer mold being formed with at least one groove on an inner surface thereof; injecting a plastic material into the groove to shape into a plastic layer which is engaged with the second face of the elastic body; removing the outer mold and the inner mold from the elastic body to provide a kinesio brace.

### BACKGROUND OF THE INVENTION

### Field of the Invention

The present invention is a CIP of application Ser. No. 15/620315, filed Jun. 12, 2017, the entire contents of which are hereby incorporated by reference.

### Description of the Prior Art

A conventional kinesio protector is tape-shaped for adhering around the joint of human for protection and support. However, it is inconvenient to use and is easy to fall off.

Brace-shaped protector can prevent the disadvantages above because it's reusable, washable, and easy to position. However, the protector is made by a single material and has a constant flexibility. Some protectors have plastic layers on the positions corresponding to joints for better support, as shown in patent TWM398951.

However, the plastic material is usually fixed on the protector by high frequency hardening. Thus, it's difficult to position the plastic material on a precise position, and the plastic material is not firmly fixed. Besides, the plastic material has to be a single piece which is large enough for high frequency hardening process. That is, if the plastic material includes plural small pieces, the plastic material cannot be fixed by the conventional means.

### SUMMARY OF THE INVENTION

The main object of the present invention is to provide a kinesio brace whose functional plastic layer is firmly engaged, and to manufacture a kinesio brace quickly.

To achieve the above and other objects, the method of manufacturing kinesio brace of the present invention includes the following steps.

Prepare an elastic body wherein the elastic body is substantially tube-shaped to have a first face inside and a second face outside. The elastic body defines an axial direction.

Insert an inner mold into the elastic body so that the first face faces to the inner mold and that the second face is opposite to the inner mold.

Placing the inner mold with the elastic body into an outer mold and close the outer mold. The outer mold has an internal space whose contour corresponds to a shape of the inner mold. The outer mold is formed with at least one groove on an inner surface thereof.

Inject a plastic material into the groove to shape into a plastic layer which is engaged with the second face of the elastic body.

Removing the outer mold from the inner mold and the elastic body and further remove the inner mold from the elastic body to provide a kinesio brace.

To achieve the above and other objects, the kinesio brace of the present invention is manufactured by the method mentioned above and includes the elastic body and the plastic layer.

Therefore, the kinesio brace can be manufactured easily, and the plastic layer is formed at the desired position firmly.

The present invention will become more obvious from the following description when taken in connection with the accompanying drawings, which show, for purpose of illustrations only, the preferred embodiment(s) in accordance with the present invention.

### BRIEF DESCRIPTION OF THE DRAWINGS

FIG. 1 is a flow chart of the present invention;

FIGS. 2-8 are stereograms of the present invention;

FIG. 9 is a stereogram showing a second embodiment of the present invention.

### DETAILED DESCRIPTION OF THE PREFERRED EMBODIMENTS

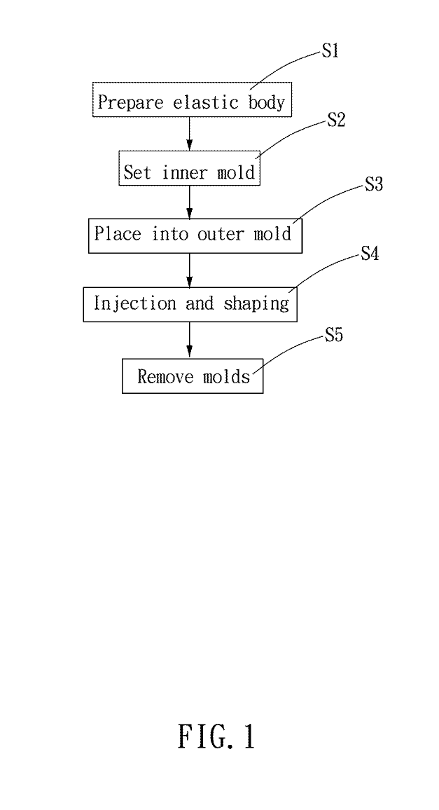

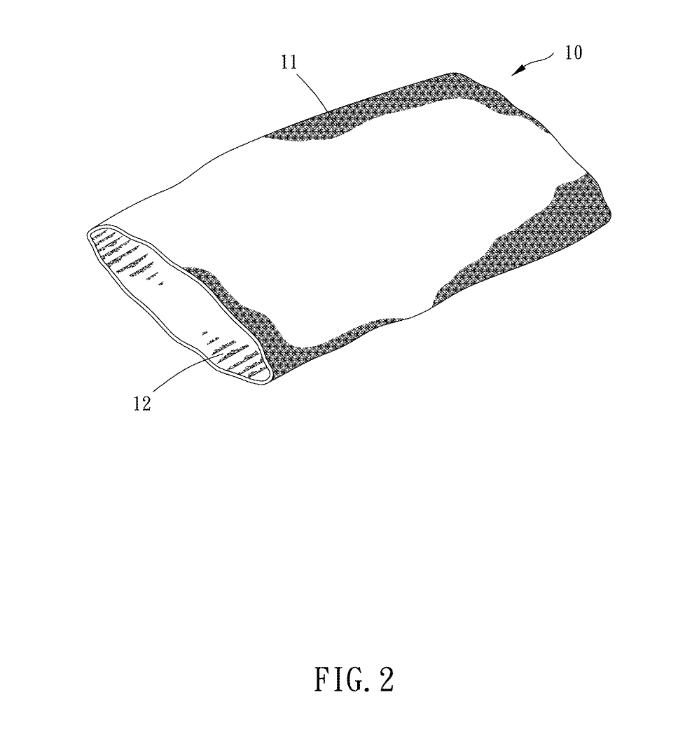

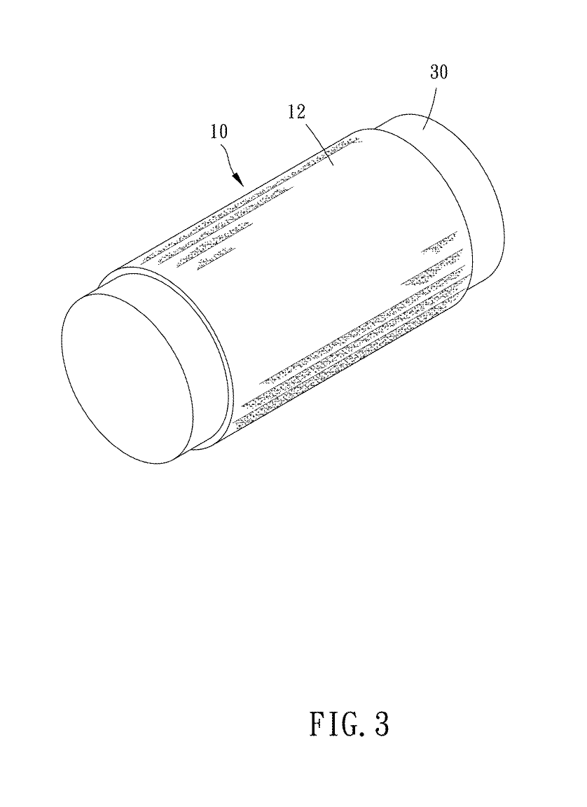

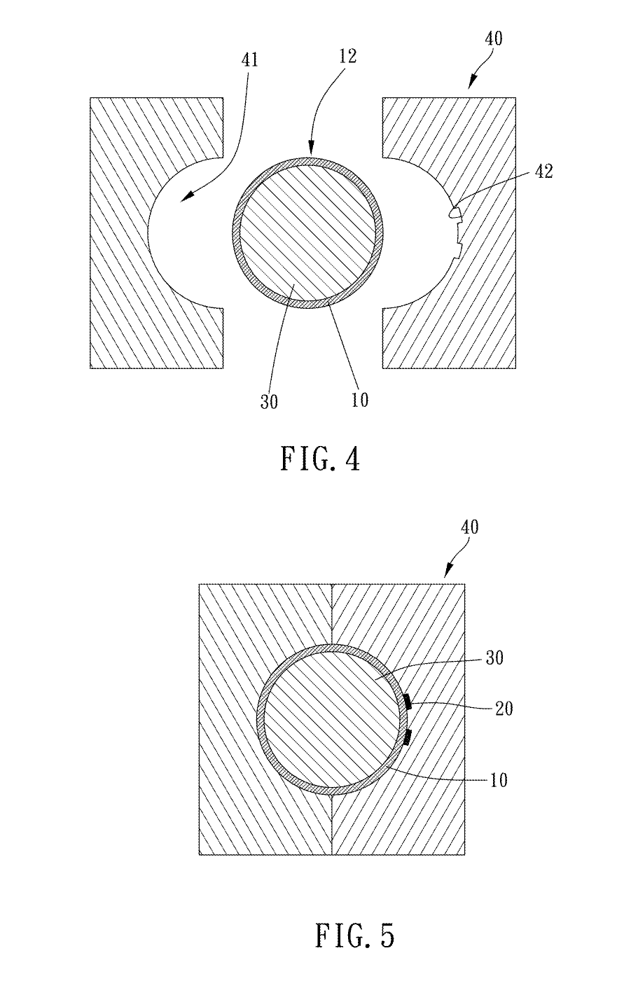

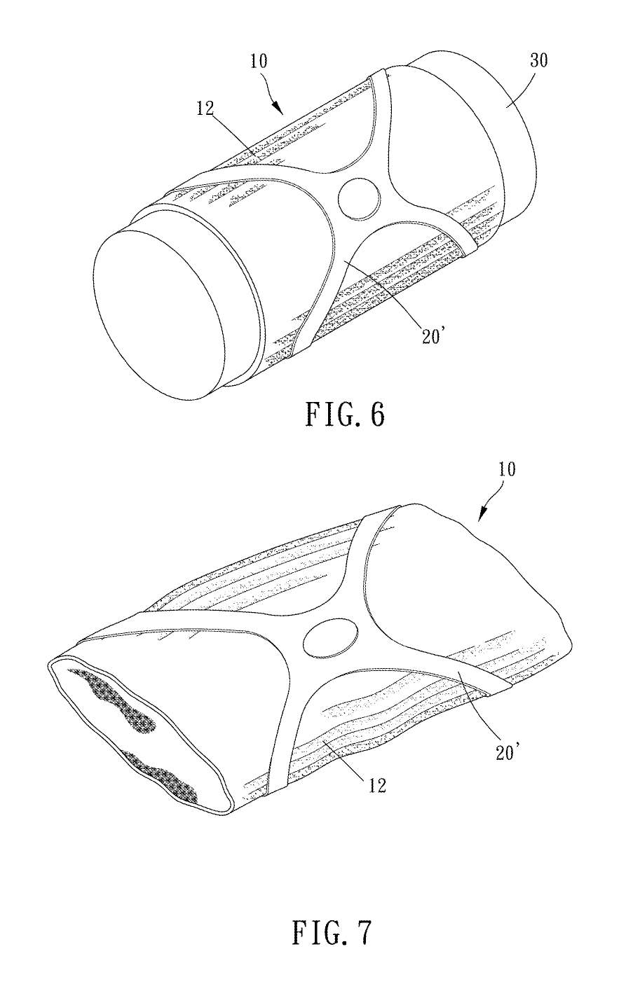

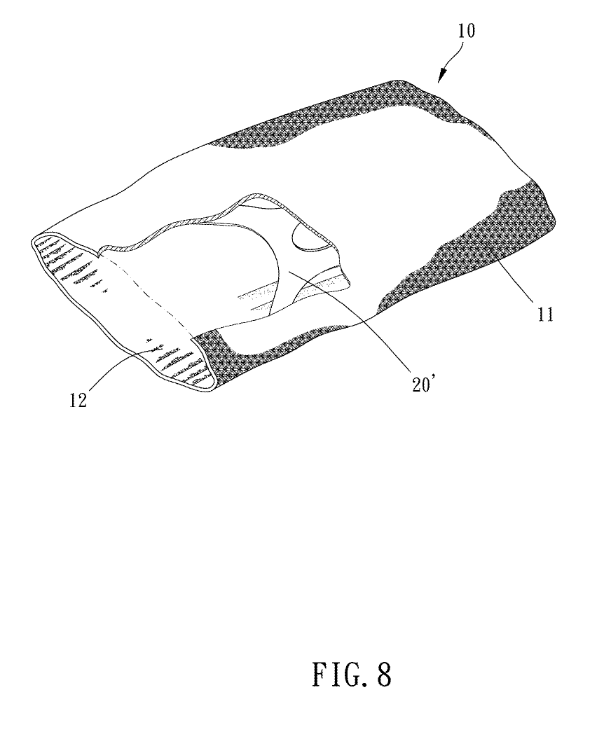

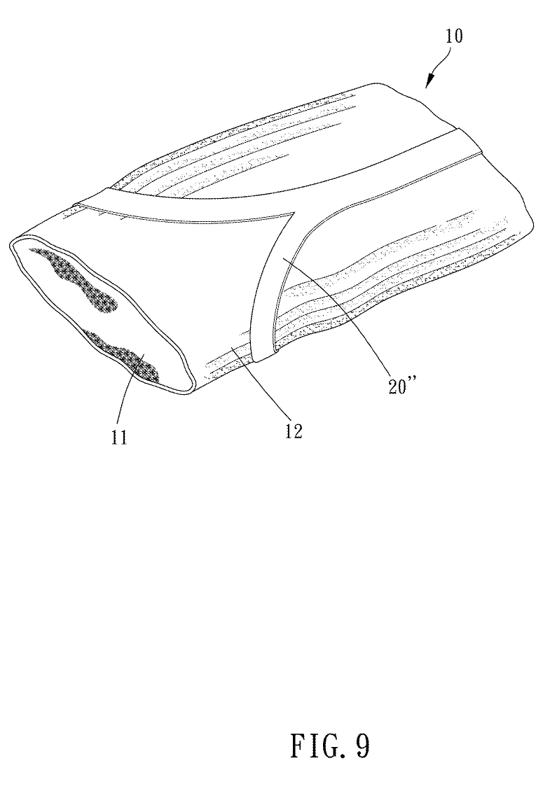

Please refer to FIGS. 1 to 9, the method of manufacturing kinesio brace of the present invention includes the following steps.

Prepare elastic body S1: Prepare an elastic body 10 wherein the elastic body 10 is substantially tube-shaped to have a first face 11 inside and a second face 12 outside. The elastic body 10 defines an axial direction. Specifically, the elastic body 10 is selected from Nylon, polyester, rubber, and Spandex.

Set inner mold S2: Insert an inner mold 30 into the elastic body 10 so that the first face 11 faces to the inner mold 30 and that the second face 12 is opposite to the inner mold 30. Thereby, the shape of the elastic body is fixed to facilitate the following processes.

Place into outer mold S3: Placing the inner mold 30 with the elastic body 10 into an outer mold 40 and close the outer mold 40. The outer mold 40 has an internal space 41 whose contour corresponds to a shape of the inner mold 30. The outer mold 40 is formed with at least one groove 42 on an inner surface thereof. Practically, the outer mold can include plural molds combined as the outer mold. Preferably, plurality of grooves 42 are formed on the outer mold 40. The grooves can be non-parallel and cross to each other. In other possible embodiments, the grooves can be parallel to each other. That is, the grooves can be arranged in the desired directions for purpose.

Injection and shaping S4: Inject a plastic material 20 into the groove 42 to shape into a plastic layer 20′,20″ which is engaged with the second face 12 of the elastic body 10. Specifically, the plastic material 20 can be TPU, TPR, or TPE.

Remove molds S5: Removing the outer mold 40 from the inner mold 30 and the elastic body 10 and further remove the inner mold 30 from the elastic body 10. Reverse the elastic body 10 to make the first face 11 faces outward so that the second face 12 and the plastic layer 20′,20″ face inward to provide a kinesio brace. Preferably, the plastic layer 20′,20″ is located at an intermediate portion of the elastic body 10 along the axial direction, and the plastic layer has a hollow portion whose position corresponds to a position of a joint of human for protection and support. It is noted that the step of reversing the elastic body 10 can be ignored to provide a kinesio brace directly, as shown in FIG. 7.

The present invention also provides a kinesio brace manufactured by the method mentioned above. The kinesio brace includes the elastic body 10 and the plastic layer 20′,20″.

What is claimed is:

1. A method of manufacturing kinesio brace, including steps of:
preparing an elastic body wherein the elastic body is substantially tube-shaped to have a first face inside and a second face outside, the elastic body defining an axial direction;
inserting an inner mold into the elastic body so that the first face faces to the inner mold and that the second face is opposite to the inner mold;
placing the inner mold with the elastic body into an outer mold and closing the outer mold, the outer mold having an internal space whose contour corresponds to a shape of the inner mold, the outer mold being formed with at least one groove on an inner surface thereof;
injecting a plastic material into the groove to shape into a plastic layer which is engaged with the second face of the elastic body;
removing the outer mold from the inner mold and the elastic body, and further removing the inner mold from the elastic body to provide a kinesio brace.

2. The method of manufacturing kinesio brace of claim 1, wherein the elastic body is made of a material selected from a group composed of nylon, polyester, rubber, and Spandex.

3. The method of manufacturing kinesio brace of claim 1, wherein the plastic material is selected from a group composed of TPU, TPR, and TPE.

4. The method of manufacturing kinesio brace of claim 1, wherein the outer mold is formed with a plurality of said grooves.

5. The method of manufacturing kinesio brace of claim 4, wherein the grooves are non-parallel and cross to each other.

6. The method of manufacturing kinesio brace of claim 1, the plastic layer is located at an intermediate portion of the elastic body along the axial direction.

7. The method of manufacturing kinesio brace of claim 1, wherein the plastic layer has a hollow portion whose position corresponds to a position of a joint of human.

8. The method of manufacturing kinesio brace of claim 1, further including a step of reversing the elastic body to make the first face faces outward so that the second face and the plastic layer face inward after removing the outer mold and the inner mold.

9. A kinesio brace, manufactured by the method of claim 1, including said elastic body and said plastic layer.
